(19) United States
(12) Patent Application Publication
(10) Pub. No.: US 2022/0273224 A1
(43) Pub. Date: Sep. 1, 2022

(54) Detection of Brief Episodes of Atrial Fibrillation

(71) Applicants: Ricardo Salinas-Martinez, Bologna (IT); Johannes de Bie, Monte San Pietro (IT); Nicoletta Marzocchi, Bologna (IT); Frida Sandberg, Lund (SE)

(72) Inventors: Ricardo Salinas-Martinez, Bologna (IT); Johannes de Bie, Monte San Pietro (IT); Nicoletta Marzocchi, Bologna (IT); Frida Sandberg, Lund (SE)

(21) Appl. No.: 17/681,460

(22) Filed: Feb. 25, 2022

Related U.S. Application Data
(60) Provisional application No. 63/154,586, filed on Feb. 26, 2021.

(51) Int. Cl. A61B 5/361; A61B 5/366; A61B 5/339; A61B 5/00

(52) U.S. Cl. CPC A61B 5/361; A61B 5/366; A61B 5/339; A61B 5/7264

(57) ABSTRACT

Systems and methods for detecting brief episodes of atrial fibrillation are described. The methods may comprise receiving from one or more sensors, data including ECG information, generating preprocessed data based on the ECG information, generating, based at least in part on the preprocessed data, a visual illustration associated with the ECG information, the visual illustration including a first section associated with a first time resolution and a second section associated with a second time resolution, receiving, as an output of a neural network, an indication of whether the visual illustration corresponds to a classification of atrial fibrillation, and assigning the visual illustrations to a classification based at least in part on the indication.

### CROSS REFERENCE TO RELATED APPLICATION

This application is a Nonprovisional of, and claims priority to, U.S. Provisional Patent Application No. 63/154,586, filed Feb. 26, 2021, the entire disclosure of which is incorporated herein by reference.

### TECHNICAL FIELD

The present application relates to systems and methods for detecting brief episodes of atrial fibrillation.

### BACKGROUND

Atrial fibrillation (AF) is the most common heart rhythm disorder found in clinical practice and is a progressive arrhythmia for which even brief episodes may represent a risk of health complications, including thrombus formation, stroke, and death. Brief episodes of AF (e.g., episodes lasting under 1 minute), may progress into longer AF episodes, resulting in increased risk of health complications. There are two main characteristics of AF, (1) heartbeat rhythm becomes irregular and (2) an absence of a P wave.

Accordingly, monitoring patients at risk of stroke and/or stroke patients may be recommended to determine the presence of brief AF episodes. However, monitoring and/or reviewing large volumes of electrocardiogram (ECG) data is time consuming, costly, and may not be accurate (e.g., due to reviewer's subjectivity).

One solution that has been proposed is using an electrocardiomatrix (ECM) to visualize long-term ECG recordings. The ECM presents the information from an ECG recording in a compact two-dimensional form, while preserving morphology and rhythm characteristics of the ECG data. The ECM considers alignment of R peaks in the ECG recordings, making it easier to evaluate whether they are preceded by P waves and/or to determine rhythm present in long-term recordings. However, review of ECMs generally remains manual, resulting in increased costs, as well as issues with accuracy (e.g., due to reviewer's subjectivity, etc.).

Deep learning (DL) approaches have also been proposed for automatic detection of brief episodes of AF. The deep learning approaches take advantage of publicly available annotated ECG databases to train DL models. However, these approaches are resource intensive and lack transparency regarding which features are used for classification, resulting in inaccurate and/or biased DL models.

Examples of the present disclosure are directed toward overcoming the issues noted above.

### SUMMARY

In an example of the present disclosure, a system comprises a processor, one or more sensors operably connected to the processor, a display operably connected to the processor, and non-transitory computer-readable media. The one or more non-transitory computer-readable media can store instructions that, when executed by the processor, cause the processor to perform operations comprising: cause the one or more sensors to capture electrocardiogram (ECG) information over a period of time, identify a plurality of time windows that are sequential and associated with the ECG information, wherein each time window of the plurality of time windows is within the period of time, create preprocessed data by truncating amplitudes of pulses represented by the ECG information, identify a first pulse corresponding to a first QRS complex represented by the preprocessed data, a first portion of the preprocessed data representing an interval of time preceding ventricular activation and a second portion of the preprocessed data representing a second interval of time following the ventricular activation, identify at least a second pulse corresponding to at least a second QRS complex represented by the preprocessed data, at least a third portion of the preprocessed data representing at least a third interval of time preceding ventricular activation and at least a fourth portion of the preprocessed data representing at least a fourth interval of time following the ventricular activation, generate an ECM illustrating the first pulse vertically aligned with at least the second pulse, generate an ECM image based on the ECM, the ECM image illustrating a first time resolution corresponding to the first portion of the preprocessed data and the third portion of the preprocessed data, input the ECM image into a neural network model configured to generate outputs indicating whether ECM images indicate atrial fibrillation, receive, based on inputting the ECM image, an indication of whether the ECM image indicates atrial fibrillation, and output, to a display, a report based at least in part on the indication.

In yet another example of the present disclosure, a method comprises receiving from one or more sensors, data including ECG information, generating preprocessed data based on the ECG information, generating, based at least in part on the preprocessed data, a visual illustration associated with the ECG information, the visual illustration including a first section associated with a first time resolution and a second section associated with a second time resolution, receiving, as an output of a neural network, an indication of whether the visual illustration corresponds to a classification of atrial fibrillation, and assigning the visual illustrations to a classification based at least in part on the indication.

The details of one or more embodiments are set forth in the accompanying drawings and the description below. Other features, objects, and advantages of these embodiments will be apparent from the description, drawings, and claims.

### DESCRIPTION OF THE FIGURES

The present invention may comprise one or more of the features recited in the appended claims and/or one or more of the following features or combinations thereof. Additionally, in this specification and drawings, features similar to or the same as features already described may be identified by reference characters or numerals which are the same as or similar to those previously used. Similar elements may be identified by a common reference character or numeral, with suffixes being used to refer to specific occurrences of the element.

FIG. 1 shows a schematic diagram of an example system for detecting brief episodes of AF.

FIG. 2 illustrates an example of time windows associated with the system of FIG. 1.

FIGS. 3A-3C illustrate example user interfaces associated with the system of FIG. 1.

FIGS. 4A-4C illustrate example user interfaces illustrating AF associated with the example system of FIGS. 1-3.

FIG. 5 illustrates an example method associated with the example system of FIGS. 1-4.

FIG. 6 illustrates a schematically illustrates an example computing device associated with the example system of FIG. 1.

### DETAILED DESCRIPTION

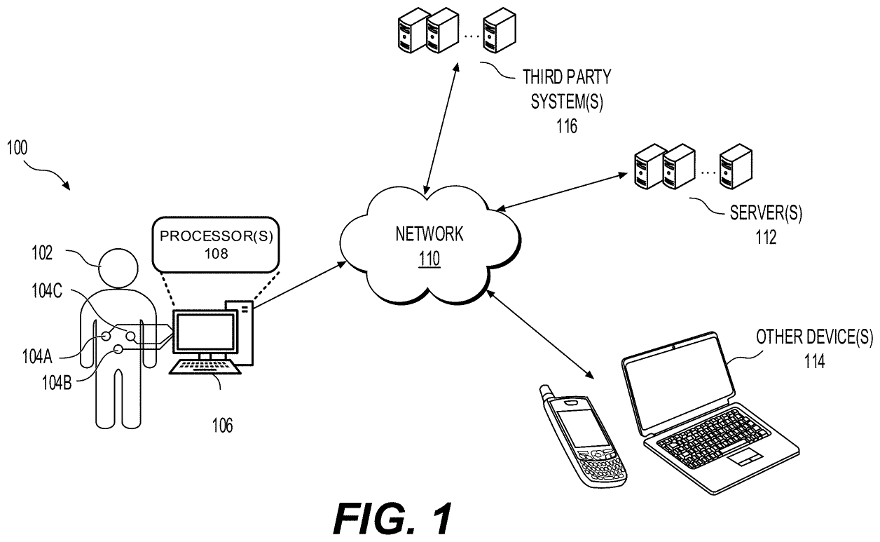

FIG. 1 is a schematic diagram of an example system 100 for detecting brief episodes of AF. In some examples, the system 100 comprises one or more servers, computing devices, and/or a cloud-based system. In some examples, the system 100 is located within a facility, such as a healthcare facility. In other examples, the system 100 is located remote from a healthcare facility.

As illustrated in FIG. 1, the system 100 may comprise one or more sensors 104 configured to sense or otherwise determine one or more medical parameters of a patient 102. In such examples, the system 100 may also include one or more computing devices 106 having one or more processors 108. In some examples, the sensor(s) 104 comprise one or more ECG leads configured to monitor heart rate of the patient 102. The sensor(s) 104 may be configured to communicate the detected ECG information (e.g., heart rate, waveform information, pulse(s), amplitude(s) associated with pulse(s) (e.g., QRS complex(es) (e.g., ventricular activation, combination of the Q wave, R wave, and S wave)), etc.) to the computing device 106 and/or processor(s) 108. The computing device 106 may comprise any computing device, network device, and/or user device (e.g., mobile device, laptop, etc.).

As illustrated in FIG. 1, the computing device 106 comprises a processor 108 (also referred to herein as a “processing unit”). Processor 108 comprises an electronic processor that operates in a logical fashion to perform operations, execute control algorithms, store and retrieve data, and other desired operations. The processor 108 may include and/or access memory, secondary storage devices, processors, processors, and any other components for running an application. The memory and secondary storage devices may be in the form of read-only memory (ROM) or random-access memory (RAM) or integrated circuitry that is accessible by the processor. Various other circuits may be associated with the system processor 108 such as power supply circuitry, signal conditioning circuitry, driver circuitry, and other types of circuitry.

Processor 108 may be a single processor or may include more than one processor. In examples where the processor 108 includes more than one processor, the processor 108 may, for example, include additional processors configured to control various functions and/or features of the system 100. As used herein, the term “processor” is meant in its broadest sense to include one or more processors, controllers, central processing units, and/or microprocessors that may be associated with the system 100, and that may cooperate in controlling various functions and operations of the system 100. The functionality of the processor 108 may be implemented in hardware and/or software without regard to the functionality. The processor 108 may rely on one or more data maps, look-up tables, neural networks (such as deep learning neural networks, convolution neural networks (CNN), etc.), algorithms, machine learning algorithms, and/or other components relating to the operating conditions and the operating environment of the system 100 that may be stored in the memory of the processor 108. Each of the data maps, look-up tables, neural networks, and/or other components noted above may include a collection of data in the form of tables, graphs, and/or equations to maximize the performance and efficiency of the system 100 and its operation.

As illustrated in FIG. 1, the computing device 106 and/or processor 108 may communicate with one or more server(s) 112 and/or other device(s) 114 via a network 110. Network(s) 110 comprise any type of wireless network or other communication network known in the art. In some examples, the network 110 comprises a local area network (“LAN”), a WiFi direct network, wireless LAN (“WLAN”), personal area network (“PAN”), virtual PAN (“VPAN”), a larger network such as a wide area network (“WAN”), cellular network connections, or a collection of networks, such as the Internet. Protocols for network communication, such as TCP/IP, 802.11a, b, g, n and/or ac, are used to implement the network 110. Although embodiments are described herein as using a network 110 such as the Internet, other distribution techniques may be implemented that transmit information via memory cards, flash memory, or other portable memory devices.

The server(s) 112 may comprise any computing device, network device, etc. In some examples, the server(s) 112 may be located at a same location (e.g., such as within the same facility, room, etc.) as the computing device 106. In some examples, the server(s) 112 may be remotely located from the computing device 106. The other device(s) 114 may comprise any computing device, user device, etc. In some examples, the other device(s) 114 correspond to a nurse's station and/or device associated with a care provider (e.g., such as a beeper, mobile device, etc. of a nurse, doctor, or other provider). In some examples, the computing device 106 and/or processor(s) 108 may generate and send alerts to the other device(s) 114 regarding a status of the patient 102. For instance, the alert may indicate, in near real-time, that the patient is experiencing an episode of AF.

As illustrated in FIG. 1, the processors 108 and/or server(s) 112 may communicate with one or more third party system(s) 116 via the network 110. The third party system(s) 116 may comprise public database(s), cloud(s), or other third party servers. For instance, the processor(s) 108 may train one or more neural network model(s) using information (e.g., ECM images, etc.) accessed from the third party system(s) 116 via the network 110.

In some examples, the system 100 may perform any of the image analysis techniques described herein using a computing model, such as a machine learning (ML) model. As used herein, the terms “machine learning,” “ML,” and their equivalents, may refer to a computing model that can be optimized to accurately recreate certain outputs based on certain inputs. In some examples, the ML models include deep learning models, such as convolutional neural networks (CNN). The term Neural Network (NN), and its equivalents, may refer to a model with multiple hidden layers, wherein the model receives an input (e.g., a vector) and transforms the input by performing operations via the hidden layers. An individual hidden layer may include multiple “neurons,” each of which may be disconnected from other neurons in the layer. An individual neuron within a particular layer may be connected to multiple (e.g., all) of the neurons in the previous layer. A NN may further include at least one fully-connected layer that receives a feature map output by the hidden layers and transforms the feature map into the output of the NN.

As used herein, the term “CNN,” and its equivalents and variants, may refer to a type of NN model that performs at least one convolution (or cross correlation) operation on an input image and may generate an output image based on the convolved (or cross-correlated) input image. A CNN may include multiple layers that transforms an input image (e.g., an ECM image) into an output image and/or output indication (e.g., such as whether the ECM image indicates AF or not (e.g., is “normal”)) via a convolutional or cross-correlative model defined according to one or more parameters. The parameters of a given layer may correspond to one or more filters, which may be digital image filters that can be represented as images (e.g., 2D images). A filter in a layer may correspond to a neuron in the layer. A layer in the CNN may convolve or cross correlate its corresponding filter(s) with the input image in order to generate the output image and/or output indication. In various examples, a neuron in a layer of the CNN may be connected to a subset of neurons in a previous layer of the CNN, such that the neuron may receive an input from the subset of neurons in the previous layer, and may output at least a portion of an output image by performing an operation (e.g., a dot product, convolution, cross-correlation, or the like) on the input from the subset of neurons in the previous layer. The subset of neurons in the previous layer may be defined according to a “receptive field” of the neuron, which may also correspond to the filter size of the neuron.

The system 100 and/or processor(s) 108 may include an ML model that is pre-trained based on training images that depict AF, images that are “normal”, as well as indications that the training images depicted (e.g., such as tags indicating which images indicate AF and which are normal). For example, one or more expert graders may review the training images and indicate whether they identify the features in the training images. Data indicative of the training images, as well as the gradings by the expert grader(s), may be used to train the ML models. The ML models may be therefore trained to identify the features in the images generated by the processor(s) 108.

In some examples, the processor 108 may cause the sensor(s) 104 to capture data during a capture window (e.g., 10 seconds, 10 hours, 24 hours, or any other suitable period of time). The processor(s) 108 may process the data using a proprietary algorithm to generate processed data. The processed data may comprise time stamp(s) associated with a same location in each wave form associated with each heartbeat. The processed data may further comprise a number assigned to each heartbeat (e.g., 1, 2, 3, 4, etc.) indicating a position in the sequence of heartbeats during the capture window. The processor(s) 108 may generate, based on the processed data, time window(s). Each time window may include a particular number of heartbeats (e.g., 10, 20, or any other suitable threshold) and/or a particular portion of the capture window (e.g., such as 10 seconds). A time window may include information starting at 0.5 seconds before the first heartbeat and ending at 3 seconds after the 10^{th} heartbeat. One or more of the time window(s) may include overlapping heartbeats. For instance, a first time window may comprise heartbeats 1-10 and the second time window may comprise heartbeats 6-15. By overlapping heartbeats within the time windows, the system 100 may detect brief AF episodes with a higher accuracy. In some examples, the number of heartbeats that overlap may be more or less than 5.

In some examples, the processor(s) 108 may pre-condition the processed data. For instance, the processor(s) 108 may truncate the processed data (e.g., ECG signal) in a time window according to one or more thresholds. For instance, the processor(s) 108 may truncate the processed data, such that waveform information above 1 millivolt (MV) and below −1 MV is removed. By removing excess waveform information, the amount of data sent and/or processed by a neural network model is reduced, which may result in improved performance of the network 110. Moreover, truncating the processed signal data may emphasize the portion of the processed data associated with a P wave, which may result in improved accuracy of identifying AF by the neural network model. The processor(s) 108 may precondition the processed data using additional or alternative techniques, such a taking an absolute value of each heartbeat signal, removing waveform information following a first heartbeat, or any other suitable technique.

In some examples, the processor(s) 108 may generate an electrocardiomatrix (ECM) by vertically aligning the portions derived from preconditioned and processed data for each time window on the first pulse identified in each portion. The processor(s) 108 may generate an ECM image (also referred to herein as a visual illustration) by converting the aligned waveforms of a time window in the ECM to a color image. For instance, the processor(s) 108 may associate an amplitude with a particular color and/or brightness. In some examples, the ECM image is decimated into to portions. For instance, a first portion of the ECM image may be associated with a first portion of the ECM, such as the first column that corresponds to information associated with 0.5 seconds before the first heartbeat that the ECM waveforms align to. This first portion of the ECM image may include an expanded time resolution (e.g., such as 2× the time resolution of the second portion of the ECM image). In this way, the information associated with absence or presence of a P wave may be emphasized.

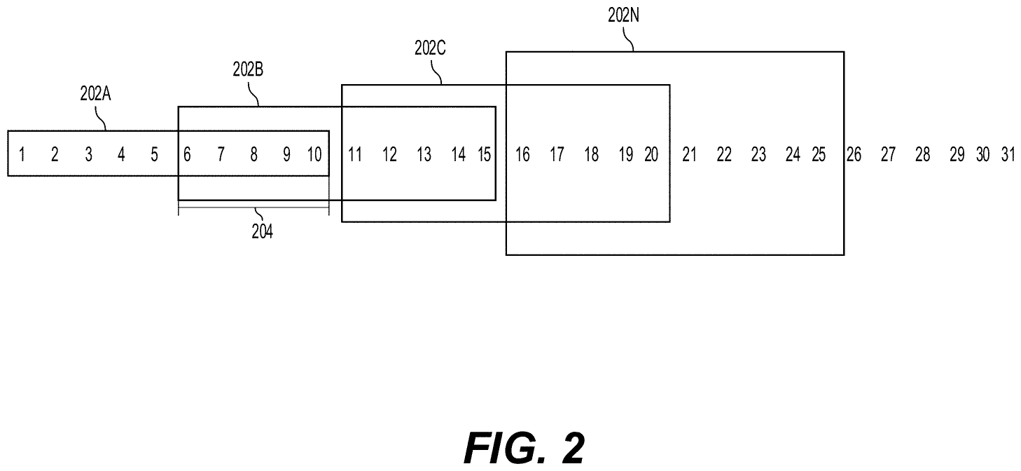

In some examples, the processor(s) 108 may input the ECM image(s) into a neural network model. The neural network model may comprise a CNN as described above and may output an indication about whether the ECM image(s) correspond to AF or not (e.g., the ECM image is “normal”). In some examples, the processor(s) 108 may assign a tag to the ECM image and/or time window based on the output of the neural network model. As described in greater detail with regard to FIG. 2 below, the processor(s) 108 may characterize and/or assign indications to individual heartbeats within a time window based on the output of the neural network model. The processor(s) 108 may additionally concatenate time windows that sequentially indicate AF and/or normal heartbeats.

The processor(s) 108 may determine, for each detected episode of AF during the capture window, the data is collected, a start time and an end time. The processor(s) 108 may make this determination based on the characterization and/or indication(s) associated with each heartbeat, as well as the time stamp associated with each heartbeat. In this way, time associated with an AF episode is tracked, such that brief episodes of AF and/or long episodes of AF may be identified.

The processor(s) 108 may generate report(s) associated with the collected data. For instance the report(s) may indicate one or more of a percentage of time during the capture window that the patient 102 was experiencing AF, a listing of times associated with AF during the capture window, an average heart rate associated with the AF episode(s), average heartrate outside of the AF episodes, among other information. In some examples, one or more of the report(s) may include table(s), graph(s), an interactive display element that enables a care provider to edit the report (e.g., accept an episode of AF, delete an episode of AF, play back data associated with the capture window, etc.), among other things.

Accordingly, the methods described herein also enable brief episodes of AF to be detected using neural network models, while also increasing accuracy of the neural network models. Thus, episodes of AF may be more accurately identified, resulting in better care for patients.

FIG. 2 illustrates an example of time windows associated with ECG information collected by the system 100 of FIG. 1. For instance, the ECG information may correspond to ECG data captured by the sensor(s) 104 described in FIG. 1 above. In some examples, the time windows 202 are generated by the processor(s) 108 of the computing device 106. In other examples, the ECG data is transmitted to the server(s) 112 via the network 110 for processing and/or generating the time windows.

As illustrated in FIG. 2, the ECG data indicates 31 pulses, amplitudes, and/or heart beats of a patient 102 (illustrated as 1-31). For instance, the processor(s) 108 may pre-process the ECG data and associate a number to each heartbeat, where the number indicates the heartbeat's sequential position in the time period and/or capture window. In some examples, the processor(s) 108 may access and/or utilize an algorithm to perform the processing. The algorithm may additionally associate time stamps with each heartbeat, where the time stamps correspond to a same location within the ECG data for each heartbeat recorded.

Once processed, the processor(s) 108 may generate the time windows illustrated in FIG. 2. In the illustrative example, each time window 202 corresponds to a portion of the capture window (e.g., such as 10 heartbeats plus 3 seconds, or any other suitable time window), with a 5 heartbeat overlap. For instance, a first time window 202A corresponds to heartbeats 1-10, a second time window corresponds to heartbeats 6-15, a third time window corresponds to heartbeats 11-20. The overlap 204 between the first time window 202A and the second time window 202B corresponds to heartbeats 6-10. In some examples and as described in greater detail below, the system 100 receives indications, for each time window 202, whether the time window is associated with AF or not. In this example, based on the indication, each heartbeat in a time window is assigned a value indicating whether or not AF and/or arrythmia is present. For instance, the system may receive an indication that the first time window 202A does not correspond to AF, a second indication that the second time window 202B does indicate AF, and a third indication that the third time window 202C does indicate AF. IN this example, the processor(s) 108 may assigned tags and/or associate indications with each individual heartbeat. For instance, the processor(s) 108 may associate beats 1-5 as being “normal” (e.g., do not indicate AF) and heartbeats 6-15 as being AF. The processor(s) 108 may concatenate heartbeats from multiple consecutive windows that either (i) indicate AF and/or (ii) do not indicate AF. Where an indication of AF is received and associated with a particular time window, the processor(s) 108 may determine that heartbeats in subsequent time windows 202 do not indicate AF (e.g., the episode of AF has ended), once two or more subsequent, consecutive indications have been received indicating there is no AF.

Accordingly, by overlapping heartbeats assigned to each time window, the techniques described herein may detect brief episodes of AF more accurately. Moreover, by concatenating time windows, the techniques described herein may provide information related to the duration and frequency of brief AF episodes and/or long AF episodes.

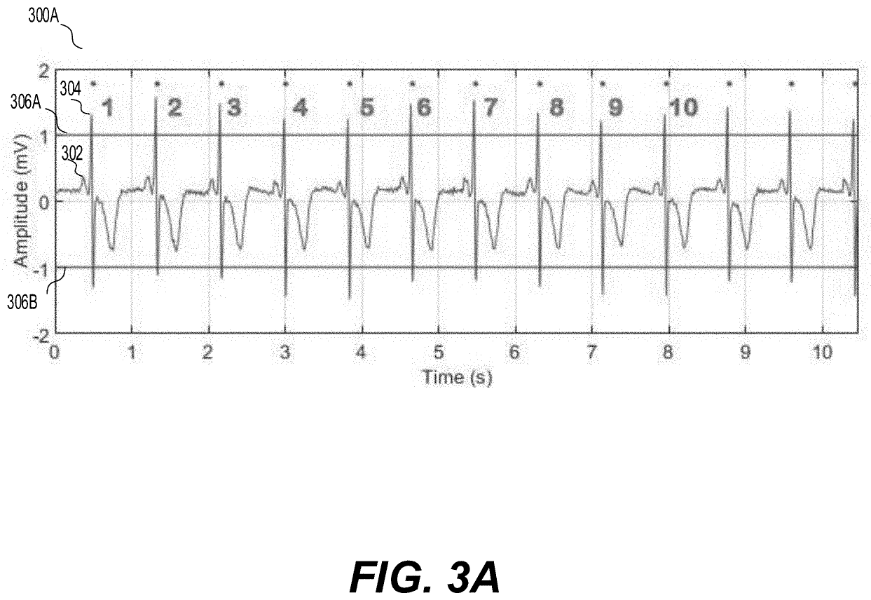

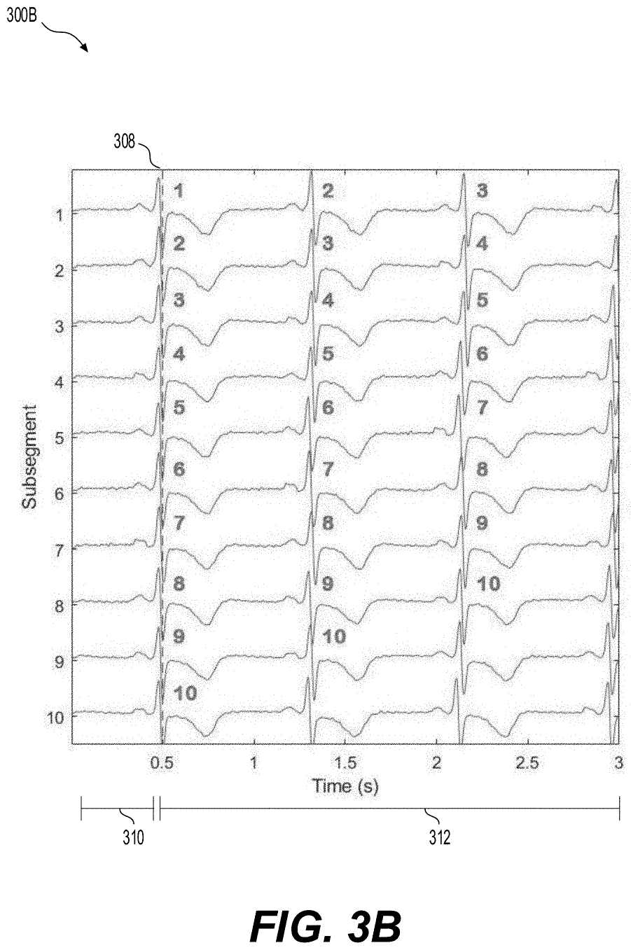

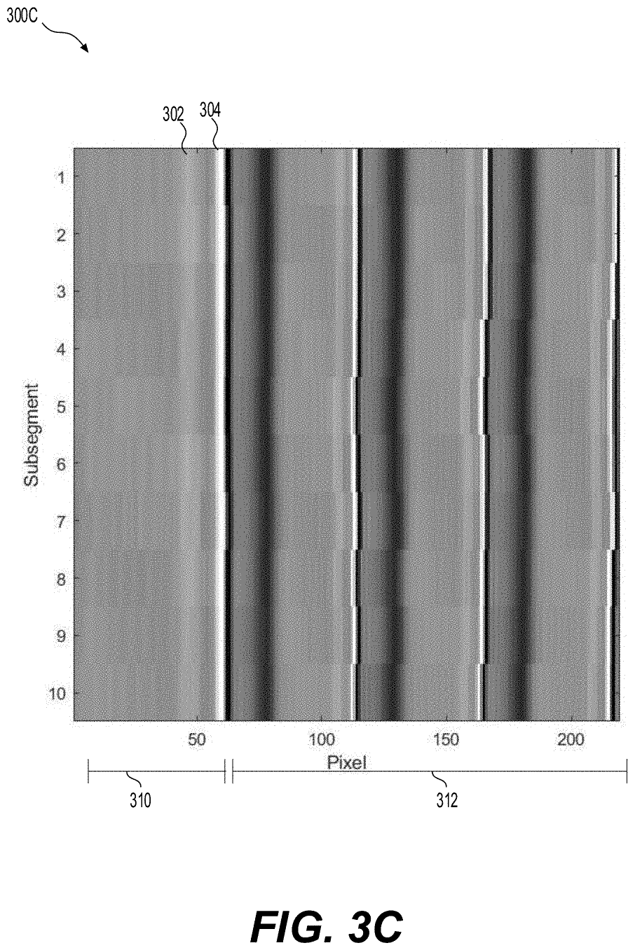

FIGS. 3A-3C illustrate example user interfaces associated with the system 100 described in FIG. 1. The user interfaces described herein may be based on one or more templates generated by the computing device of the system 100. Additionally or alternatively, the user interfaces described herein may be generated by the one or more computing devices and/or device(s) based at least in part on instructions received from the system. As discussed above, the user interfaces described herein may, but need not, be implemented in the context of the system 100.

FIG. 3A illustrates an example user interface 300A in accordance with some embodiments of the present disclosure. In some examples, user interface 300A is displayed and/or presented by the computing device 106. For instance, the user interface 300A may display and/or present ECG information in real-time. In some examples, the user interface 300A may be associated with server(s) 112 and/or other device(s) 114. As illustrated, the user interface 300A may include ECG information that corresponds to a waveform of a heartbeat of a patient 102. The ECG information may include waveform information, including a P wave 302 and a QRS complex 304, or any other ECG information.

The ECG information (e.g., waveform information, pulse(s), amplitude(s), QRS complex(es), P wave(s), etc.) may be transformed into an ECM image (as shown in FIG. 3C). To transform the ECG information, the processor(s) 108 may pre-condition the ECG information. For instance, as described above, pre-conditioning may comprise truncating the ECG information, such that waveform and/or QRS complex information above a first threshold 306A and below a second threshold 306B in FIG. 3A is removed. Additionally, or alternatively, pre-conditioning the ECG information may comprise taking absolute values of the signal (e.g., QRS complex) associated with each heartbeat and/or preserving ECG information for a time period (e.g., 0.5 seconds prior to heartbeat 1, or any other suitable time period) prior to a first heartbeat in the time window, removing waveform information after the first peak (e.g., QRS information 304), and only including time information associated with the peaks of the heartbeats after the first peak 304.

FIG. 3B illustrates an example user interface 300B of an electrocardiomatrix (ECM). As noted above, an ECM presents the information from an ECG recording in a compact two-dimensional form, while preserving morphology and rhythm characteristics of the ECG data. The ECM considers alignment of R peaks in the ECG recordings, making it easier to evaluate whether they are preceded by P waves and/or to determine rhythm present in long-term recordings. The illustrative example in FIG. 3B corresponds to processed and pre-conditioned data (e.g., labelled, time stamped, and truncated ECG and/or waveform information) for a time window of 10 heartbeats (+3 seconds). As described above the heartbeats in the time window are labelled as 1-10. As noted above, each heartbeat captured during a capture window may be processed and associated with a time stamp. The time stamp may correspond to a same portion of the waveform of the individual heartbeat. As illustrated in FIG. 3B the x-axis corresponds to time and covers an interval associated with a portion of the time window (e.g., 0.5 seconds prior to the first heartbeat and +2.5 seconds after the first heartbeat). The y-axis corresponds to the subsegment of heartbeats included in the portion of the time window. As described above, the waveforms of heartbeats 1-3 are placed in row 1 of the ECM, with waveform information 0.5 seconds prior to the first heartbeat and 2.5 seconds after the first heartbeat. Row 2 corresponds to heartbeats 2-4 and includes waveform information corresponding to the 0.5 seconds prior to the 2^{nd} heartbeat and 2.5 seconds after the second heartbeat. As illustrated in FIG. 3B, this stacking pattern is repeated until all of the first 10 heartbeats in the time window are aligned at an alignment point 308. As described above, the alignment point 308 of the heartbeats may be based on the time stamps corresponding to the same portion of the waveform of the heartbeat. Accordingly, each heartbeat in a first column of the ECM is aligned at a same point (e.g., alignment point 308) in the waveform. Accordingly, any misalignment of the heartbeats in subsequent columns of the ECM may indicate an irregular pattern associated with AF. Moreover, by aligning the first heartbeats at the alignment point 308, the portion of waveform information in the 0.5 seconds prior to the heartbeat peak (e.g., QRS complex 304 described in FIG. 3A above) is once again emphasized, enabling easier identification of presence and/or absence of a P wave. Alternatively, pre-conditioning the ECG information may comprise preserving ECG information for a time period around the first heartbeat in each row (e.g., 0.5 seconds prior and 0.5 seconds after, or any other suitable time period), removing waveform information after the first peak (e.g., QRS complex 304), and only including time information associated with the peaks of the heartbeats after the first peak 304.

FIG. 3C illustrates an example user interface 300C corresponding to a visual illustration of the ECM described in FIG. 3B. For instance, the visual illustration (also referred to herein as an ECM image) may be directly generated based on the ECM from FIG. 3B. For instance, each row in FIG. 3C may correspond to a row in the ECM of FIG. 3B (e.g., row 1 in FIG. 3C corresponds to the waveform information in row 1 of FIG. 3B, etc.). As noted above, the processor(s) 108 may generate an ECM image by converting the aligned waveforms of a time window in the ECM to a color image. For instance, the processor(s) 108 may associate an amplitude with a particular color and/or brightness. Each row in an ECM may be converted to generate an ECM image in FIG. 3C. In some examples, the ECM image is decimated into to portions. For instance, a first portion 310 of the ECM image may be associated with a first portion of the ECM, such as the information associated with 0.5 seconds before the alignment point 308 of the first heartbeat that the ECM waveforms align to. This first portion 310 of the ECM image may include an expanded time resolution (e.g., such as 2× the time resolution of the second portion 312 of the ECM image). In the illustrative example of FIG. 3C, the expanded time resolution on the x-axis is represented in pixels. For instance, the first portion 310 of the visual illustration may have a higher time resolution than the second portion 312 of the visual illustration. In the illustrative example, FIG. 3C shows the presence of a P wave information is illustrated by the item 302 (e.g., corresponding to a light gray column). The QRS complexes 304 of the aligned heartbeats in the are illustrated by item 304 (e.g., corresponding to the white and black columns). Subsequent P waves can be seen as aligned in the second portion of the visual illustration, along with subsequent QRS complexes, indicating the heartbeat is “normal”. Accordingly, by using an expanded time resolution on the first portion of the visual illustration, the information associated with absence or presence of a P wave may be emphasized. Moreover, by aligning the QRS complexes, any irregularity in heartbeat may be shown in the second portion 312 of the visual illustration.

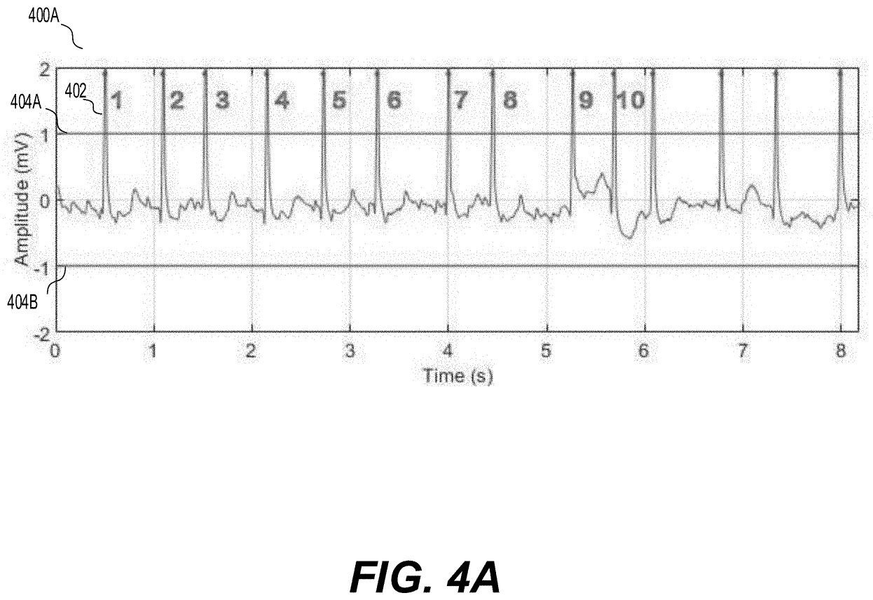

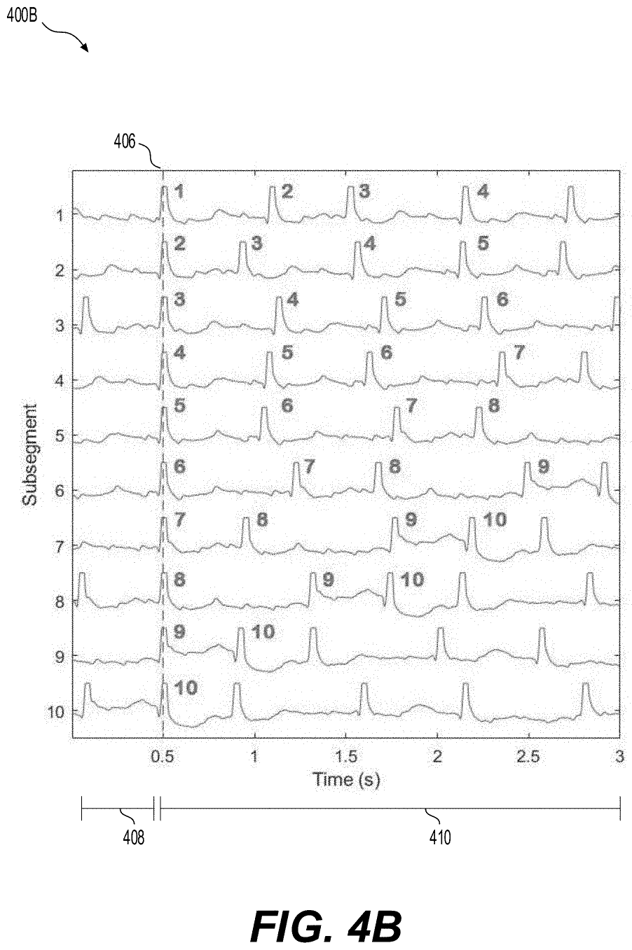

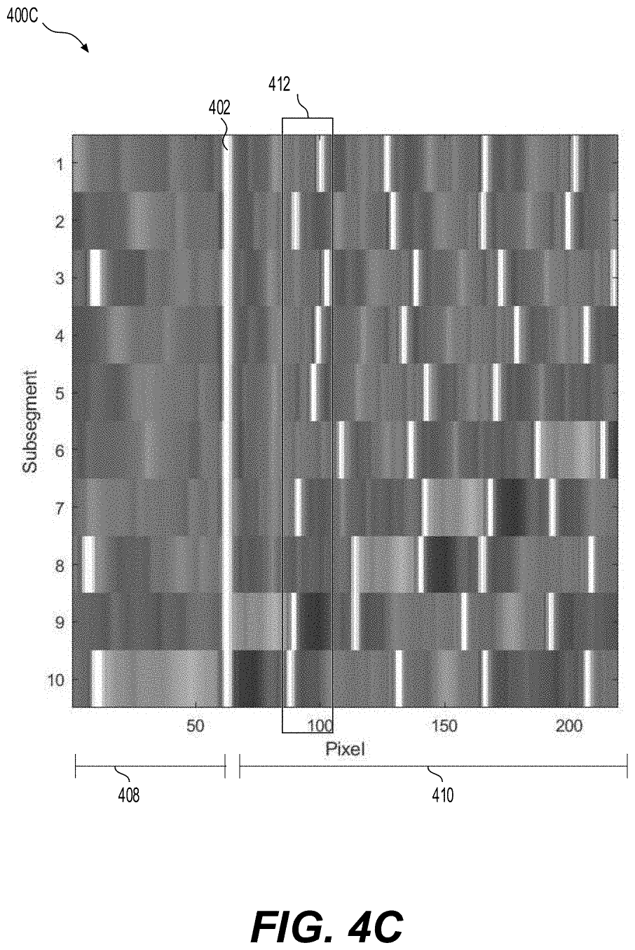

FIGS. 4A-4C illustrate example user interfaces associated with the system 100 described in FIG. 1. The user interfaces described herein may be based on one or more templates generated by the computing device of the system 100. Additionally or alternatively, the user interfaces described herein may be generated by the one or more computing devices and/or device(s) based at least in part on instructions received from the system. As discussed above, the user interfaces described herein may, but need not, be implemented in the context of the system 100. In some examples, FIGS. 4A-4C represent user interfaces associated with ECG information that indicates AF.

FIG. 4A illustrates an example user interface 400A in accordance with some embodiments of the present disclosure. In some examples, user interface 400A is displayed and/or presented by the computing device 106. For instance, the user interface 400A may display and/or present ECG information in real-time. In some examples, the user interface 400A may be associated with server(s) 112 and/or other device(s) 114. As illustrated, the user interface 400A may include ECG information that corresponds to a waveform of a heartbeat of a patient 102. The ECG information may include waveform information, such as a QRS complex 402.

The ECG information (e.g., waveform information, pulse(s), amplitude(s), QRS complex(es), P wave(s), etc.) may be transformed into an ECM image (as shown in FIG. 4C). To transform the ECG information, the processor(s) 108 may pre-condition the ECG information. For instance, as described above, pre-conditioning may comprise truncating the ECG information, such that waveform and/or QRS complex information above a first threshold 404A and below a second threshold 404B in FIG. 4A is removed.

FIG. 4B illustrates an example user interface 400B of an ECM. As noted above, an ECM presents the information from an ECG recording in a compact two-dimensional form, while preserving morphology and rhythm characteristics of the ECG data. The ECM considers alignment of R peaks in the ECG recordings, making it easier to evaluate whether they are preceded by P waves and/or to determine rhythm present in long-term recordings. The illustrative example in FIG. 4B corresponds to processed and pre-conditioned data (e.g., labelled, time stamped, and truncated ECG information and/or waveform information) for a time window of 10 heartbeats (+about 3 seconds). As described above the heartbeats in the time window are labelled as 1-10. As noted above, each heartbeat captured during a capture window may be processed and associated with a time stamp. The time stamp may correspond to a same portion of the waveform of the individual heartbeat. As illustrated in FIG. 4B the x-axis corresponds to time and covers a portion of the time window. The y-axis corresponds to the subsegment of heartbeats included in the portion of the time window. As described above, the waveforms of heartbeats 1-3 are placed in row 1 of the ECM, with waveform information 0.5 seconds prior to the first heartbeat, indicated by item 408, and 2.5 seconds after the first heartbeat, indicated by item 410. Row 2 corresponds to heartbeats 2-4 and includes waveform information corresponding to the 0.5 seconds prior to the 2^{nd} heartbeat and 2.5 seconds after the second heartbeat. As illustrated in FIG. 4B, this stacking pattern is repeated until all of the first 10 heartbeats in the time window are lined up at the alignment point 406. As described above, the alignment point 406 may be based on the time stamps corresponding to the same portion of the waveform of the heartbeat. Accordingly, each heartbeat is aligned at a same point (e.g., alignment point 406) in the waveform. Accordingly, any misalignment of the heartbeats in subsequent columns of the ECM may indicate an irregular pattern associated with AF. Moreover, by aligning the first heartbeats in a first column of the ECM at the alignment point 406, the portion of waveform information in the 0.5 seconds prior to the heartbeat peak (e.g., QRS complex 402 described in FIG. 4A above) is once again emphasized, enabling easier identification of presence and/or absence of a P wave. Alternatively, pre-conditioning the ECG information may comprise preserving ECG information for a time period around the first heartbeat in each row (e.g., 0.5 seconds prior and 0.5 seconds after, or any other suitable time period), removing waveform information after the first peak (e.g., QRS complex 402), and only including time information associated with the peaks of the heartbeats after the first peak 402.

FIG. 4C illustrates an example user interface 400C illustrating another example of a visual illustration associated with an ECM. In some examples, the visual illustration of FIG. 4 corresponds to an example of a visual illustration that indicates AF. For instance, the first portion 408 of the visual illustration in FIG. 4C corresponds to the 0.5 seconds prior to the first peak that the heartbeats in a time window are aligned on. As illustrated in FIG. 4C, there is no light grey band prior to the QRS complex 402, indicating in this case the absence of P waves prior to the aligned peaks. Moreover, the amplitudes and/or time stamps associated with the subsequent QRS complexes of the heartbeats are not aligned, as indicated by item 412. Accordingly, a neural network model may determine, based on the visual illustration of FIG. 4C, that the heartbeats in the particular time window correspond with an indication of AF.

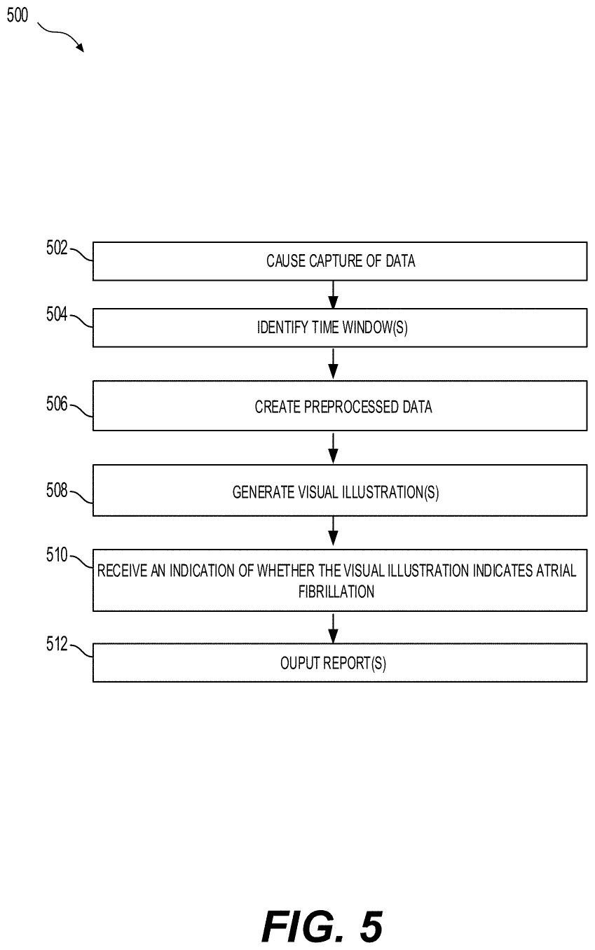

FIG. 5 illustrates an example method 500 associated with the system shown in FIGS. 1-4. The example method 500 is illustrated as a logical flow graph, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be combined in any order and/or in parallel to implement the processes. Although any of the processes or other features described with respect to the methods 400 and/or 500 may be performed by processor(s) 108 and/or the server(s) 112, for ease of description, the example method 500 will be described below as being performed by the processor 108 of the computing device 106 unless otherwise noted.

As illustrated in FIG. 5, at 502, the processor(s) 108 may cause the capture of data. For instance, the processor(s) 108 may cause the sensor(s) 104 to capture data associated with a patient 102 for a particular capture window (e.g., 10 seconds, 10 hours, 24 hours, or any other suitable period of time. In some examples, the data comprises ECG information.

In some examples, the processor(s) 108 may process the data. For instance, the processor(s) 108 may process the data (e.g., number each heartbeat, associated time stamps with each heartbeat, etc.) using a proprietary algorithm to generate processed data. The processed data may comprise time stamp(s) associated with a same location in each wave form associated with each heartbeat. The processed data may further comprise a number assigned to each heartbeat (e.g., 1, 2, 3, 4, etc.) indicating a position in the sequence of heartbeats during the capture window.

At 504, the processor(s) 108 may identify a plurality of time window(s) that are sequential. For instance, the processor(s) 108 may generate, based on the processed data, time window(s). Each time window may include a particular number of heartbeats (e.g., 10, 20, or any other suitable threshold). A time window may include information starting at 0.5 seconds before the first heartbeat and ending at 2.5 seconds after the last selected heartbeat. One or more of the time window(s) may include overlapping heartbeats. For instance, a first time window may comprise heartbeats 1-10 and the second time window may comprise heartbeats 6-15. By overlapping heartbeats within the time windows, the system 100 may detect brief AF episodes with a higher accuracy. In some examples, the number of heartbeats that overlap may be more or less than 5.

At 506, the processor(s) 108 may create preprocessed data. In some examples, creating the pre-processed data may comprise truncating the processed data (e.g., ECG signal) in a time window according to one or more thresholds. For instance, the processor(s) 108 may truncate the processed data, such that waveform information and/or amplitude information for one or more pulses above a first threshold (.e.g., 1 millivolt (mV) or any other suitable threshold) and below a second threshold hold (e.g., below −1 mV or any other suitable threshold) is removed. By removing excess waveform information, the amount of data sent and/or processed by a neural network model is reduced, which may result in improved performance of the network 110. Moreover, truncating the processed data may emphasize the portion of the processed data associated with a P wave, which may result in improved accuracy of identifying AF by the neural network model. The processor(s) 108 may create the pre-processed data using additional or alternative techniques, such a taking an absolute value of each heartbeat signal, removing waveform information following a first heartbeat, or any other suitable technique.

In some examples, the processor(s) 108 may identify one or more pulse(s) represented by the preprocessed data, where portion(s) of the preprocessed data represent presence or absence of P wave associated with the respective pulse(s). For instance, the processor(s) 108 may identify a defined number of pulses (QRS complexes) represented by the preprocessed data, portions of the preprocessed data representing possible atrial activity (P wave) preceding each pulse, and portions representing the intervals of time following each pulse. These portions of the preprocessed data may be used to generate an ECM and/or ECM image.

At 508, the processor(s) 108 may generate one or more visual illustration(s). For instance, the processor(s) 108 may generate an electrocardiomatrix (ECM) using the preprocessed data for each time window. For instance, as noted above, the ECM may illustrate one or more pulse(s) vertically aligned at a same location and/or position in the waveform(s) of a time window. The processor(s) 108 may then generate a visual illustration by converting the aligned waveforms of a time window in the ECM to a color image (e.g., an ECM image), as described above with regard to FIGS. 3B and 3C. For instance, the processor(s) 108 may associate an amplitude with a particular color and/or brightness. Each row in an ECM may be converted to generate the visual illustration. In some examples, the visual illustration is decimated into to portions and/or sections. For instance, a first portion and/or section of the visual illustration may be associated with a first portion and/or section of the ECM, such as the first column that corresponds to information associated with 0.5 seconds before the first heartbeat that the ECM waveforms align to. This first portion and/or section of the visual illustration may include an expanded time resolution (e.g., such as 2× the time resolution of the second portion of the visual illustration). In this way, the information associated with absence or presence of a P wave may be emphasized.

At 510, the processor(s) 108 may receive an indication of whether the visual illustration indicates atrial fibrillation. For instance, the processor(s) 108 may input the visual illustration into a neural network model. The neural network model may comprise a CNN as described above and may output an indication about whether the visual illustration correspond to AF or not (e.g., the visual illustration is “normal”). In some examples, the processor(s) 108 may assign a tag to the visual illustration and/or corresponding time window based on the output of the neural network model. As described in FIG. 2, the processor(s) 108 may characterize and/or assign indications to individual heartbeats within a time window based on the output of the neural network model. The processor(s) 108 may additionally concatenate time windows that sequentially indicate AF and/or normal heartbeats. In some examples, the processor(s) 108 may input each visual illustration generated for each of the one or more time windows into the neural network model.

In some examples, the processor(s) 108 may determine whether a visual illustration indicates AF or not by using machine learning mechanisms and using information associated with a plurality of patients, procedures, etc. Such machine-learning mechanisms include, but are not limited to supervised learning algorithms (e.g., artificial neural networks, Bayesian statistics, support vector machines, decision trees, classifiers, k-nearest neighbor, etc.), unsupervised learning algorithms (e.g., artificial neural networks, association rule learning, hierarchical clustering, cluster analysis, etc.), semi-supervised learning algorithms, deep learning algorithms (e.g., a CNN), etc.), statistical models, etc. In at least one example, machine-trained data models can be stored in memory associated with the processor 108.

At 512, the processor(s) 108 may output report(s). In some examples, the processor(s) 108 may determine, for each detected episode of AF during the capture window, the data is collected, a start time and an end time. The processor(s) 108 may make this determination based on the characterization and/or indication(s) associated with each heartbeat, as well as the time stamp associated with each heartbeat. In this way, time associated with an AF episode is tracked, such that brief episodes of AF and/or long episodes of AF may be identified. The processor(s) 108 may generate report(s) based at least in part on the indications output by the neural network model and the determinations of AF start and end times. For instance the report(s) may indicate one or more of a percentage of time during the capture window that the patient 102 was experiencing AF, a listing of times associated with AF during the capture window, an average heart rate associated with the AF episode(s), average heartrate outside of the AF episodes, among other information. In some examples, one or more of the report(s) may include table(s), graph(s), an interactive display element that enables a care provider to edit the report (e.g., accept an episode of AF, delete an episode of AF, play back data associated with the capture window, etc.)., among other things.

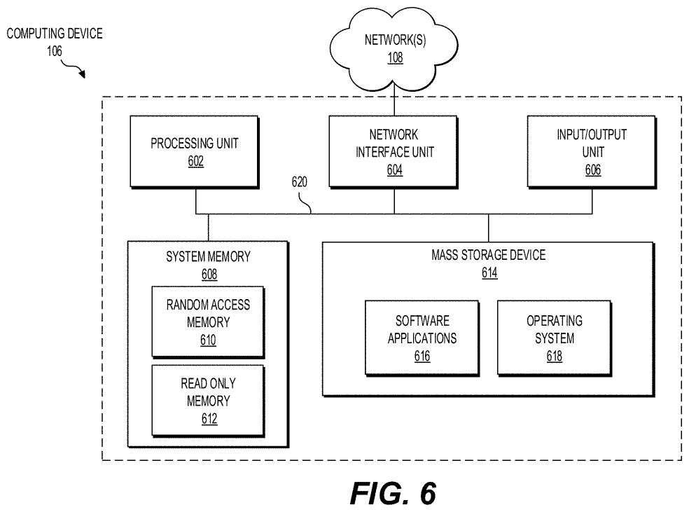

FIG. 6 schematically illustrates an example computing device 106 used to implement aspects of the present disclosure. The processor architecture shown in FIG. 6 illustrates any type of computing device 106 and/or processor 106, and/or any type of computing device, such as a conventional server computer, workstation, desktop computer, laptop, tablet, network appliance, e-reader, smartphone, or other computing device, and can be utilized to execute any of the software components presented herein. The computing device 106 may, in some examples, correspond to any device described herein, and may comprise personal devices (e.g., smartphones, tables, wearable devices, laptop devices, etc.) networked devices such as servers, switches, routers, hubs, bridges, gateways, modems, repeaters, access points, and/or any other type of computing device that may be running any type of software and/or virtualization technology.

As illustrated in FIG. 6, the computing device 106 comprises a processing unit 602, a system memory 608, and a system bus 620 coupling the system memory 608 to the processing unit 602. Processing unit 602 comprises one or more processor(s), processor(s), at least one central processing unit (“CPU”), memory, and a system bus that couples the memory to the CPU. In some examples, the memory of the processing unit 602 includes system memory 608 and mass storage device. System memory 608 includes random access memory (“RAM”) 610 and read-only memory (“ROM”) 612. In some examples, a basic input/output system (BIOS) that contains the basic routines that help to transfer information between elements within the example computing device 106 and/or processor 108, such as during startup, is stored in the ROM 612.

In some examples, the mass storage device 614 of the processing unit 602 stores software instructions and data. In some examples, mass storage device 614 is connected to the CPU of the processing unit 602 through a mass storage processor (not shown) connected to the system bus 620. The processing unit 602 and its associated computer-readable data storage media provide non-volatile, non-transitory storage for the example computing device 106 and/or processor 108. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Although the description of computer-readable data storage media contained herein refers to a mass storage device, it should be appreciated by those skilled in the art that computer-readable data storage media can be any available non-transitory, physical device or article of manufacture from which the device can read data and/or instructions. The mass storage device 614 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the example computing device 106 and/or processor 108.

The computing device 106 may operate in a networked environment using logical connections to remote network devices, including the server(s) 112, other device(s) 114, and/or third party system(s) 116, through the network(s) 110. The computing device 106 connects to the network(s) 110 through a network interface unit 604 connected to the system bus 620. The network interface unit 604 may also be utilized to connect to other types of networks and remote computing systems.

Input/output unit 606 is configured to receive and process input from a number of input devices. Similarly, the input/output unit 606 may provide output to a number of output devices.

Mass storage device 614 and/or RAM 610 store software instructions and data. For instance, the software instructions can include an operating system 618 suitable for controlling the operation of a device. The mass storage device 614 and/or the RAM 610 also store software instructions 616, that when executed by the processing unit 602, cause the device to perform the techniques described herein.

As a result, the methods and systems described herein may assist caregivers with patient care. Additionally, by continuously monitoring event progression, etc., the techniques and systems described herein enable healthcare facilities to provide personalized care to patients. This may streamline workflow for providing care within the healthcare facility, thereby reducing costs for the patient and/or the healthcare facility.

The foregoing is merely illustrative of the principles of this disclosure and various modifications can be made by those skilled in the art without departing from the scope of this disclosure. The above described examples are presented for purposes of illustration and not of limitation. The present disclosure also can take many forms other than those explicitly described herein. Accordingly, it is emphasized that this disclosure is not limited to the explicitly disclosed methods, systems, devices, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices, and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

The description and illustration of one or more embodiments provided in this application are not intended to limit or restrict the scope of the invention as claimed in any way. Regardless whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the claimed invention and the general inventive concept embodied in this application that do not depart from the broader scope.

What is claimed is:

1. A system, comprising:
a processor;
one or more sensors operably connected to the processor;
a display operably connected to the processor; and
non-transitory computer-readable media storing instructions that, when executed by the processor, cause the processor to perform operations comprising:
cause the one or more sensors to capture electrocardiogram (ECG) information over a period of time;
identify a plurality of time windows that are sequential and associated with the ECG information, wherein each time window of the plurality of time windows is within the period of time;
create preprocessed data by truncating amplitudes of pulses represented by the ECG information;
identify a first pulse corresponding to a first QRS complex represented by the preprocessed data, a first portion of the preprocessed data representing an interval of time preceding ventricular activation and a second portion of the preprocessed data representing a second interval of time following the ventricular activation;
identify at least a second pulse corresponding to at least a second QRS complex represented by the preprocessed data, at least a third portion of the preprocessed data representing at least a third interval of time preceding ventricular activation and at least a fourth portion of the preprocessed data representing at least a fourth interval of time following the ventricular activation;
generate an ECM illustrating the first pulse vertically aligned with at least the second pulse;
generate an ECM image based on the ECM, the ECM image illustrating a first time resolution corresponding to the first portion of the preprocessed data and the third portion of the preprocessed data;
input the ECM image into a neural network model configured to generate outputs indicating whether ECM images indicate atrial fibrillation;
receive, based on inputting the ECM image, an indication of whether the ECM image indicates atrial fibrillation; and
output, to a display, a report based at least in part on the indication.

2. The system of claim 1, wherein the creating the preprocessed data further comprises taking absolute values associated with the ECG information.

3. The system of claim 1, wherein the one or more sensors comprise one or more ECG leads.

4. The system of claim 1, further comprising:
generating a second ECM image associated with another portion of the preprocessed data;
determining, based on inputting the second ECM image into the neural network model, a second indication of whether the second ECM image indicates atrial fibrillation; and
outputting, to the display, the report including the indication and the second indication.

5. The system of claim 1, wherein the ECM image comprises a first section and a second section, wherein the first section of the ECM image is associated with an expanded time resolution relative to a second time resolution associated with the second section of the ECM image.

6. The system of claim 1, wherein the ECG information comprises a plurality of pulses and identifying the plurality of time windows further comprises associating individual time stamps to a same portion of each pulse of the plurality of pulses.

7. The system of claim 6, further comprising:
determine, based at least in part on the indication from the neural network model and the time stamps, one or more start times and end times associated with one or more episodes of atrial fibrillation; and
output a listing associated with the one or more episodes of atrial fibrillation.

8. The system of claim 1, wherein the plurality of time windows comprise a first time window corresponding to a first set of pulses and a second time window corresponding to a second set of pulses, wherein the first set of pulses and the second set of pulses comprise one or more overlapping pulses.

9. The system of claim 1, wherein the plurality of time windows are each associated with a portion of the ECG information associated with a portion of the period of time.

10. A method comprising:
causing one or more sensors to capture electrocardiogram (ECG) information over a period of time;
identifying a plurality of time windows that are sequential and associated with the ECG information, wherein each time window of the plurality of time windows is within the period of time;
creating preprocessed data by truncating amplitudes of pulses represented by the ECG information;
identifying a first pulse corresponding to a first QRS complex represented by the preprocessed data, a first portion of the preprocessed data representing an interval of time preceding ventricular activation and a second portion of the preprocessed data representing a second interval of time following the ventricular activation;
identifying at least a second pulse corresponding to at least a second QRS complex represented by the preprocessed data, at least a third portion of the preprocessed data representing at least a third interval of time preceding ventricular activation and at least a fourth portion of the preprocessed data representing at least a fourth interval of time following the ventricular activation;
generating an ECM illustrating the first pulse vertically aligned with at least the second pulse;
generating an ECM image based on the ECM, the ECM image illustrating a first time resolution corresponding to the first portion of the preprocessed data and the third portion of the preprocessed data;
input the ECM image into a neural network model configured to generate outputs indicating whether ECM images indicate atrial fibrillation;
receiving, based on inputting the ECM image, an indication of whether the ECM image indicates atrial fibrillation; and
outputting, to a display, a report based at least in part on the indication.

11. The method of claim 10, further comprising:
generating a second ECM image associated with a second portion of the ECG information;
determining, based on inputting the second ECM image into the neural network model, a second indication of whether the second ECM image indicates atrial fibrillation; and
outputting, to the display, the report including the indication and the second indication.

12. The method of claim 11, further comprising:
determining that the ECM image indicates atrial fibrillation;
determining that the second ECM image indicates atrial fibrillation; and
concatenating a first time window associated with the ECM image and a second time window associated with the second ECM image.

13. The method of claim 10, wherein the ECG information comprises a plurality of pulses corresponding to a plurality of ECG signals and creating the preprocessed data further comprises associating individual time stamps to a same portion of each pulse of the plurality of ECG signals.

14. The method of claim 10, wherein creating the preprocessed data further comprises taking absolute values associated with the ECG information.

16. The method of claim 10, wherein the ECM image comprises a first section and a second section, wherein the first section of the ECM image is associated with an expanded time resolution relative to a second time resolution associated with the second section of the ECM image.

17. A method comprising:
receiving from one or more sensors, data including ECG information;
generating preprocessed data based on the ECG information;
generating, based at least in part on the preprocessed data, a visual illustration associated with the ECG information, the visual illustration including a first section associated with a first time resolution and a second section associated with a second time resolution;
receiving, as an output of a neural network, an indication of whether the visual illustration corresponds to a classification of atrial fibrillation; and
assigning the visual illustrations to a classification based at least in part on the indication.

18. The method of claim 17, wherein the visual illustration comprises an ECM image.

19. The method of claim 17, wherein the first time resolution is greater than the second time resolution.

20. The method of claim 17, wherein a first portion of the preprocessed data is associated with the first section of the visual illustration and a second portion of the preprocessed data is associated with the second section of the visual illustration.
